(19) United States
(12) Patent Application Publication
(10) Pub. No.: US 2026/0334859 A1
(43) Pub. Date: Oct. 29, 2026

(54) WEARABLE FEEDBACK SYSTEM TO LIMIT SHOULDER INJURY

(71) Applicant: West Virginia University Board of Governors on Behalf of West Virginia University, Morgantown, WV (US)

(72) Inventors: Stephen Cain, Morgantown, WV (US); Samuel White, Morgantown, WV (US); Kassandra Hoff, Morgantown, WV (US); Romeo Mitchell, Morgantown, WV (US); Blake Wolfe, Morgantown, WV (US)

(21) Appl. No.: 19/656,295

(22) Filed: Apr. 23, 2026

Related U.S. Application Data
(60) Provisional application No. 63/793,291, filed on Apr. 23, 2025.

(51) Int. Cl. A61B 5/00; A61B 5/107

(52) U.S. Cl. CPC A61B 5/4576; A61B 5/1071; A61B 5/6802; A61B 5/6824; A61B 5/7405; A61B 5/7455; A61B 5/746

(57) ABSTRACT

Various examples are provided related to a wearable feedback system to limit shoulder injury. For example, a system for a wearable feedback system to limit shoulder injury can include a wearable device configured to be positioned on a shoulder of a user. Further, the system can include at least one sensor coupled to the wearable device. Further, the system can include a processing unit operatively coupled to the at least one sensor. Further, the processing unit can be configured to determine, based on signal data received from the at least one sensor, a should elevation angle. Then, the shoulder elevation angle can be compared to a predefined elevation threshold. Finally, in response to determining that that shoulder elevation angle exceeds the predefined threshold, the at least one feedback device can be caused to generate an alert.

### CROSS REFERENCE TO RELATED APPLICATIONS

This application claims priority to, and the benefit of, co-pending U.S. provisional application entitled “Wearable Feedback System to Limit Shoulder Injury” having Ser. No. 63/793,291, filed Apr. 23, 2025, which is hereby incorporated by reference in its entirety.

### BACKGROUND

Overwhelming evidence correlates repetitive overhead work/movements to various musculoskeletal disorders. Specifically, individuals who are required to perform overhead movements during work are two to three times more likely to develop a shoulder injury than those who do not perform these movements. Furthermore, research shows that working at arm elevations of 90°or higher or above shoulder height for 10% of a standard work shift doubles injury risk alone. Shoulder angle and arm elevation were determined as the risk criteria for a wearable device for shoulder injury prevention.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of rotator cuff anatomy, in accordance with various embodiments of the present disclosure.

FIG. 2A illustrates an example of identifying that working above shoulder height is higher risk than a neutral position, in accordance with various embodiments of the present disclosure.

FIG. 2B illustrates an example of a table comparing different monitoring devices that would record varying shoulder injury factors, in accordance with various embodiments of the present disclosure.

FIGS. 3A and 3B illustrate an exemplary sensor monitoring device and a microcontroller unit (MCU), respectively, in accordance with various embodiments of the present disclosure.

FIG. 3C Illustrates exemplary prototype components, in accordance with various embodiments of the present disclosure.

FIG. 4 illustrates an exemplary flex sensor position on a user shoulder brace, in accordance with various embodiments of the present disclosure.

FIG. 5 illustrates an exemplary thermoplastic polyurethane container that can be used to house electrical components, in accordance with various embodiments of the present disclosure.

FIG. 6 illustrates an exemplary schematic for PCT fabrication, in accordance with various embodiments of the present disclosure.

FIG. 7 illustrates an example of an assembled prototype, in accordance with various embodiments of the present disclosure.

FIG. 8 illustrates an example top side view of the prototype components, in accordance with various embodiments of the present disclosure.

FIG. 9 illustrates an example back side view of the prototype components that attach to shoulder brace, in accordance with various embodiments of the present disclosure.

FIG. 10 illustrates an example prototype worn by a user, in accordance with various embodiments of the present disclosure.

FIG. 11 illustrates exemplary data logged to the microSD memory card showing the shoulder angle over a duration of five minutes measured by the flex sensor in 2 orthogonal (e.g. x- and y-) axes in accordance with various embodiments of the present disclosure.

FIG. 12 illustrates an example cycle of care for wearable shoulder injury prevention device for an individual that has already suffered an injury, in accordance with various embodiments of the present disclosure.

FIG. 13 illustrates an example flowchart in accordance with various examples of the present disclosure.

### DETAILED DESCRIPTION

Disclosed herein are various examples related to a wearable feedback system to limit shoulder injury. The use of sensors, motors, circuit boards, a battery, and compression shoulder brace is described for use in the wearable feedback system. And the use of software and a controller is described for use in connection with the wearable feedback system. A device that can detect potential shoulder injury is important because shoulder pain can affect roughly 18-26% of all adults during some point in their lifetime. In specific regards to injury, each year nearly 2 million people living in the United States visit their doctor regarding a rotator cuff tear. The rotator cuff acts as a conjunctive point of stability for the glenohumeral joint. Reference will now be made in detail to the description of the embodiments as illustrated in the drawings, wherein like reference numbers indicate like parts throughout the several views.

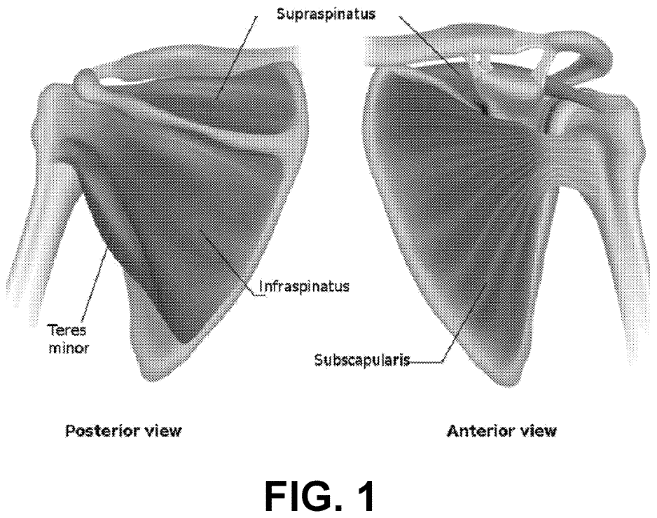

With reference to FIG. 1, shown is a diagram of rotator cuff anatomy according to various examples of the present disclosure. FIG. 1 shows four separate muscles that make up the rotator cuff: subscapularis, infraspinatus, teres minor, and supraspinatus. These rotator muscles can be responsible for protecting the shoulder during various movements an individual can conduct. During movement, they can prevent the humerus head from sliding which can ultimately allow for a complete range of motion and full stability. Like other muscles, they can be susceptible to being compromised and injured due to certain movements, postures, etc. Rotator cuff injuries can be seen mostly in three subgroups of individuals: people over the age of 40, athletes, and people who work jobs that can require them to perform consistent overhead movements. There can be a need to quantify and measure shoulder movements and decrease the chances an individual would develop a shoulder injury.

In view of the above, the various examples of the present disclosure allow for real-time measurements of a user's shoulder angle and, in turn, can correlate the position of the user's arm to their shoulder height. Further, the various examples of the present disclosure allow for the user to be notified when they perform a movement which increases the chance the user may develop a shoulder injury. For example, the various examples of the present disclosure can measure capability with feedback techniques so that the user wearing the device can be alerted when they perform an arm movement above their shoulder height and the user can be notified when they perform a movement that placed their arm above shoulder height. By identifying these movements, it is intended that the user develops healthier shoulder movements and improved shoulder health.

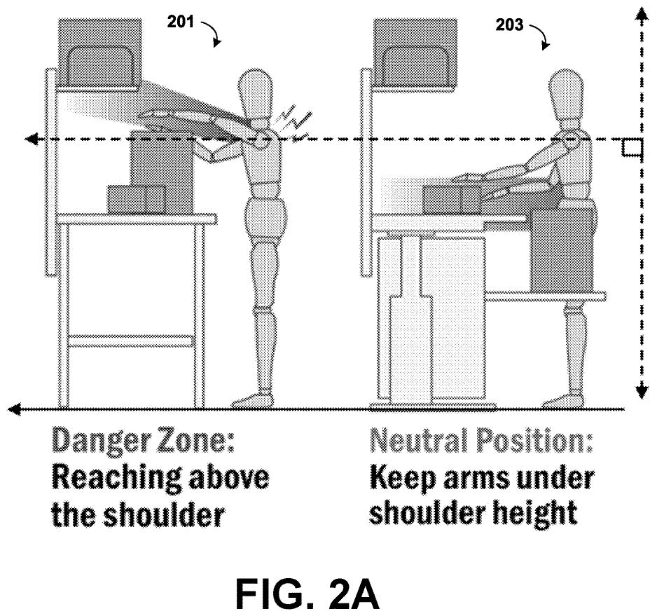

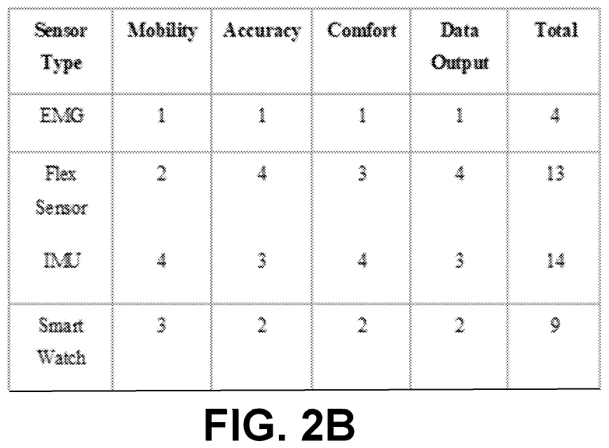

With reference to FIG. 2A, shown is an example of a diagram identifying that working above shoulder height 201 is higher risk than working at a neutral position 203, according to various examples of the present disclosure. This distinction can be important because working at arm elevations of at least 90°or above shoulder height 201 for 10% of a daily work shift can increase a person's risk for injury. This degree of arm elevation can be approximately in line with or above shoulder height 201. Considering the different ways a shoulder injury may occur, methods were identified to measure the causes of shoulder injury and develop a monitoring system that could appropriately quantify these causes with their respective data. Further, because arm elevations can be identified as a contributor to a person's risk for injury, the device was developed to measure shoulder angle/arm position and to reduce the overall risk a user has of developing a shoulder injury.

With reference to FIG. 2B, shown is an example of a table showing a ranking system that compares different monitoring devices that can record varying shoulder injury factors. The ranking system compares the sensor types according to four distinct categories: mobility, accuracy, comfort, and data output. Each sensor was given a score between one and four—with four indicating that the sensor is the most optimal in that certain field. The device needs to be practical in everyday scenarios. It should not hinder the wearer or user's ability to complete simple shoulder and arm movements. Additionally, it should be capable of being worn for long periods of time, so a certain level of comfort can be maintained for the user. Lastly, the accuracy and data output recorded from the device can be reliable and applicable to the designated criteria. The table considers the effectiveness in light of these factors for electromyography (EMG), flex sensors, inertial measurement units (IMU), and smart watches.

EMG: By using electrodes placed into the desired muscles, nerve stimulation can be used by a monitoring system to measure a muscle's electrical activity and overall response. An EMG device can be used to detect neuromuscular abnormalities.

Flex Sensor: Generic single axis flex sensors tend to measure varying resistance. When bending a flex sensor, the resistance throughout the sensor can change to determine how much the device bends. Dual axis flex sensors, such as the Nitto Denko Corporation's 2-axis sensor, measures differential capacitance. This measurement is linearly proportional to the angular displacement of the two ends of the sensor.

IMU: With the combination of accelerometers, gyroscopes, and magnetometers, these devices record angular rates, specific forces, and even surrounding magnetic fields.

Smart Watch: Like an IMU, using a combination of accelerometers, gyroscopes, and in some watches, magnetometers, this type of device can be used for similar measurements as an IMU.

The flex sensor and standalone IMU can be preferred monitoring devices compared to alternative options. Because the user may wear the device for an extended period while completing numerous arm movements, an EMG can be impractical in terms of mobility and comfort along with both accuracy and data output. For example, if the electrodes were to alter during movement, then they can dislodge and lead to inaccurate readings. Additionally, a smart watch IMU can be less preferred as it is a less accurate version of the stand alone IMU. Even though a smart watch has high practicality for everyday use, it is not considered reliable for its data output.

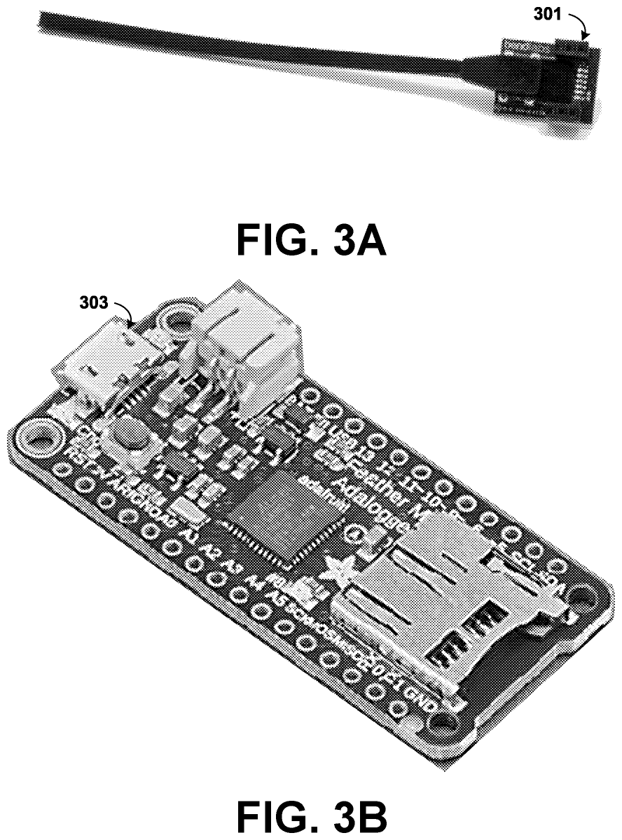

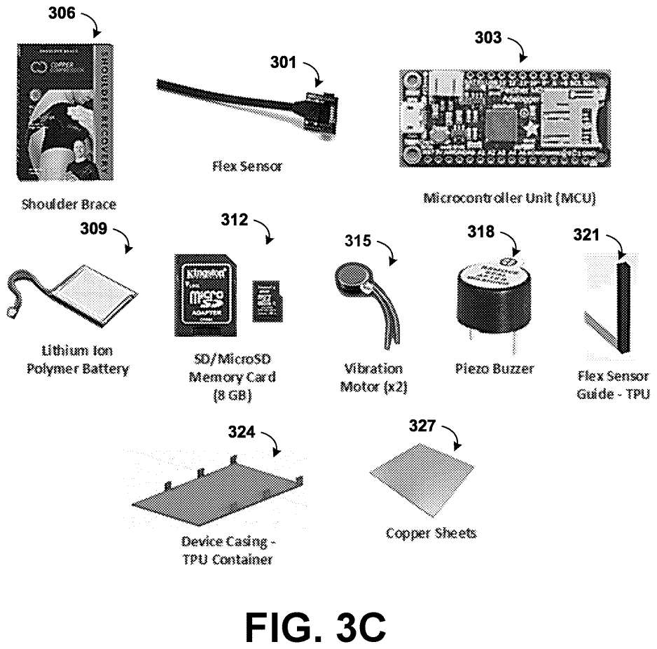

With reference to FIG. 3A shown is an example of an exemplary sensor 301 monitoring device. In some examples, the sensor 301 can be a flex sensor 301 The flex sensor 301 provides a practical approach for the monitoring device, such a flex sensor 301 is the NittoBend Technologies 2-axis soft flex sensor. Unlike a simple 1-axis flex sensor, this 2-axis sensor 301 has measuring capabilities in two orthogonal planes. It works by, for example, measuring the differential capacitance through the sensor 301 which is linearly proportional to the angular displacement of the head and tail of the device. By using this sensor 301, the angle of a user's arm can be measured if it was placed on the shoulder. Since research shows that a user working overhead for extended periods of time can lead to shoulder injury, the flex sensor 301 can determine when a user has their arms above shoulder height 201 and thus putting them at risk.

With reference to FIGS. 3B and 3C, shown is an example of a microcontroller unit (MCU) 303, the Adafruit Feather M0 Adalogger, in addition to an example of individual exemplary prototype components according to various examples of the present disclosure, respectively, in accordance with various examples of the present disclosure. In a present nonlimiting example, the primary components of a prototype device can be a NittoBend Technologies 2-axis flex sensor 301 (shown in FIG. 3A), an Adafruit Feather M0 Adalogger, a printed circuit board (PCB), two vibration motors 315, a piezo buzzer 318, a 3.7V Lithium-Ion Polymer battery 319, and a Copper Compression Shoulder Brace 306. By using the flex sensor 301 and placing it on the side of a user's shoulder, the angle of the user's arm can be measured and related to their arm's elevation. Additionally, with the use of a PCB, all electrical components can be soldered together making the device's size more compact. Then, a thermoplastic polyurethane (TPU) container 324 can be 3D printed using CAD software to contain the PCB and all other components. With the entire device excluding the battery, placed in the TPU container 324, it can be mounted onto the side of a user shoulder brace 306 using Velcro® with the flex sensor's 301 tail pointing down the user's arm.

Feedback methods can be visual, haptic, auditory, or can include other methods. This device can use haptic or auditory feedback while in use. In the present nonlimiting example, a microcontroller unit 303 that allows for such communication between them can be the Adafruit Feather M0 Adalogger (MCU). The MCU 303 can also have a power source and a memory. In some examples, the power source can be connected via an on-board JST connector that enables the use of Lithium-Ion Polymer Battery 309. Further, the MCU 303 can be connected to a memory, such as a MicroSD memory card 312 holder for proper data storage. The device can be practically contained on a user's shoulder in various arm positions while not restricting movement in any way.

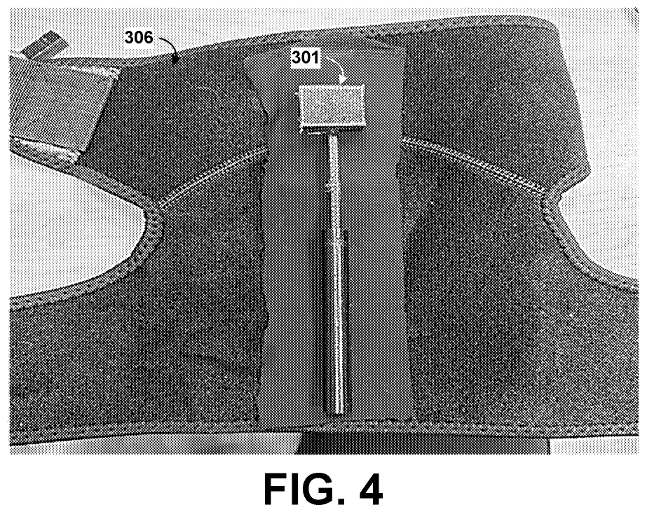

Referring next to FIG. 4, shown is an example of an exemplary flex sensor 301 position on a user's shoulder brace 306, according to various examples of the present disclosure. In some examples using a copper compression shoulder brace 306, the device can be placed on the side of the brace due to its Velcro® capabilities. To connect all the electrical components, a PCB can be fabricated so that the size of the device does not interfere with the user while wearing the brace. Each component can be soldered onto the PCB, and can allow direct connection between the MCU, the flex sensor 301, the feedback units, and two buttons for device function which are described in further detail below. The PCB and the other components can then be constrained in a 3D TPU container 324. In some examples, the 3D TPU container 324 can be 3-D printed. Using Velcro® attached to the back of the TPU container 324, the device can be placed on the side of the shoulder brace 306 to hold the entire device in a fixed position.

The MCU 303 can be used to charge the Li—Po battery 309. For example, by having a JST connector on the MCU 303, a microUSB port can be used to recharge the battery through the MCU 303. The MCU 303 can also have a MicroSD memory card 312 holder which can provide access to, for example, 8 gigabytes of data storage. This data can be reviewed and used to determine how often the user went above shoulder height 201 with their arm positions. Another aspect is the use of a 2-axis flex sensor 301. Unlike the single axis flex sensor 301 also produced by NittoBend Technologies, the 2-axis sensor 301 can measure angular displacement in two orthogonal planes instead of one. The use of a PCB can reduce device size and complexity.

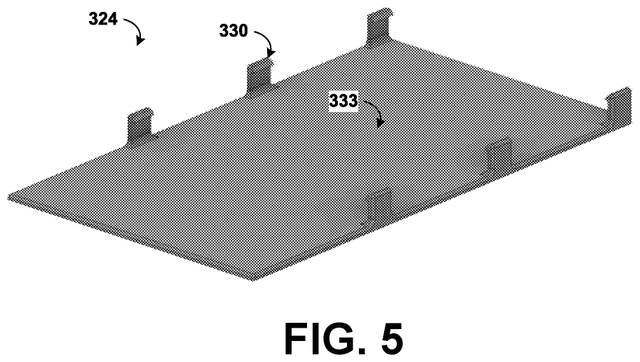

Referring next to FIG. 5, shown is an exemplary TPU container 324 that can be used to house electrical components according to various examples of the present disclosure. In the present nonlimiting example, a TPU container 324 can be designed so that the PCB can be placed into a secure and fixed position. In this example, the base 333 measures 95×52 mm and has six additional pieces that are raised 4 mm above this surface. The measurements of the base can allow for the PCB to fit precisely inside the outer edges along with the flex sensor 301 to be added at the bottom end of base. The side pieces 330 that extrude upwards can be high enough to reach over the PCB and all the components that can be soldered on top of it. This can allow for ridges to be added that overhang and can keep the PCB from falling off the base piece.

The electronic components of the device prototype can be networked with each other and interconnected all or in part via an electronic substrate. In the present nonlimiting example, the electrical components can be soldered to a custom PCB designed using, for example, the computer software EasyEDA®. The board can be printed from a fiberglass sheet plated with copper on both sides. The copper not needed can be burned off using a laser to form the wire paths on the surface of the board. The wire paths can be, for example, 0.5 mm wide and can be custom drawn to allow all paths to be on the bottom side of the board. This can allow for the copper paths and soldered connections to be protected from wear during use. A piece of foam can also placed underneath the PCB for protection and to help secure it to the 3D printed case.

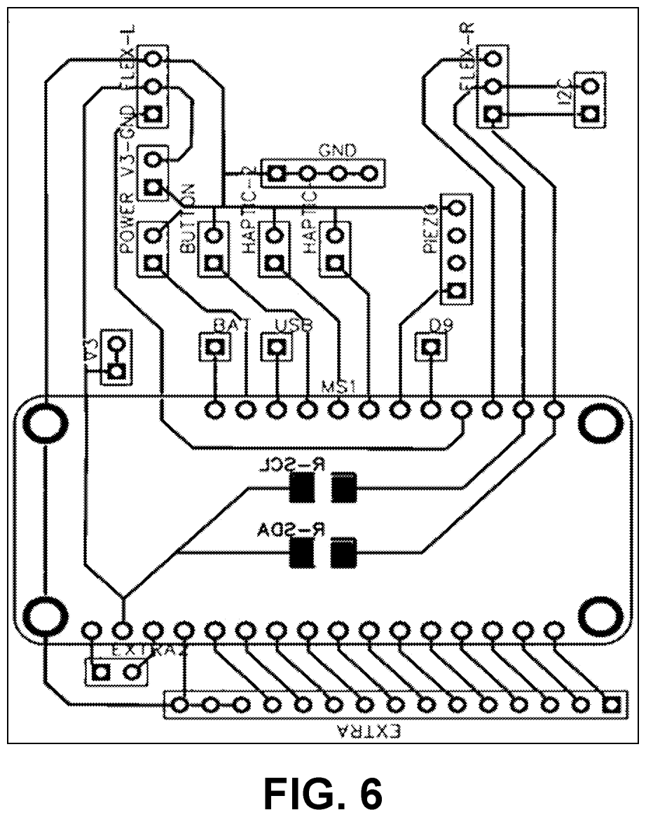

Referring next to FIG. 6, shown is an example of an exemplary schematic for PCB fabrication, according to various examples of the present disclosure. In the present nonlimiting example, the PCB can be constructed with additional connections available for potentially expanded capability. It can be designed for the MCU 303 to be directly soldered to the board and positioned to easily access the microSD memory card slot, microUSB port, and pinouts of the MCU 303. The pinholes used in the prototype can be labeled, for example, FLEX-L, FLEX-R, POWER, HAPTIC-1/2, and PIEZO. The flex sensor 301 can have six total connections that are pathed through, for example, FLEX-L and FLEX-R. The power button can be attached to the ground and the En pin on the Feather which, when connected, can turn the device off. The haptic vibration motors, button and piezo can all use one data pin and one ground pin each connected to the Feather. In some examples, the piezo buzzer 318 can be soldered directly to the PCB while the other components can use 22-gauge stranded wire run from the pinouts to their location on the 3D printed casing.

In the present nonlimiting example, the 2-axis flex sensor 301 from NittoBend Technologies can be used, as shown in FIG. 2. This flex sensor 301 is a type of deformation sensor 301 that has two axes with a 0.18°measurement resolution, and has low power consumption, flexibility, long life cycle, and data stability. It functions by measuring the angle in each axis and outputs the value as a digital signal using I2C (Inter-Integrated Circuit) communication. The I2C communication can work by having one wire running between a SDA (Serial Data) pin on the flex sensor 301 and a SDA pin of the MCU 303, and second wire running between a SCL (Serial Clock) pin on the flex sensor and a SCL pin of the MCU 303. This setup can allow the sensor 301 to communicate with the MCU 303 using only two wires and to send the collected data to be processed by the MCU 303. The flex sensor 301 can use four extra connections to function with two of those being ground and a dedicated voltage into the sensor that can function between a range of 1.8 volts and 3.63 volts. In some examples, the flex sensor 301 can be connected to a 3.3 volt regulated pin on the MCU 303. The final two connections for the sensor 301 can be a data ready line to calibrate the sensor and a line to reset the sensor 301. In some examples, calibrating the sensor 301 can involve establishing an initial reference position when the arm is at rest, such that later measurements can be determined relative to this reference.

In some examples, feedback to the user can be allowed when the shoulder angle measured by the flex sensor 301 is greater than about 90°or above shoulder height 201. In some examples, this threshold angle can be set such that the angle is any angle that a person is at increased risk for shoulder injury when their shoulder angle is above the threshold. In the present example, feedback can be provided in the form of a piezo buzzer 318 mounted directly on the PCB and two haptic vibrating motors 315 attached to the bottom of the 3D printed casing. The device can alert the user with two short feedback pulses when the measured shoulder angle from the flex sensor 301 is, for example, greater than 90°. It is set to alert the user every five seconds while their arm is above the angle.

The feedback in the present nonlimiting example can be a mix of haptic and auditory pings and can be given in four different modes based on an individual user's preference. The user can cycle between feedback methods by pressing the button to the right of the flex sensor 301 with one click moving to the next feedback. The default feedback can be set to haptic using the two vibrating mini motor discs located on the underside of the device casing that can be felt through the shoulder brace 306. In some examples, the feedback method can be auditory, using a piezo buzzer 318 mounted directly on to the top of the PCB which can emit, for example, a high pitch ping. In some examples, the feedback can be a combination of haptic and auditory feedback. In some examples, the user can not be alerted while wearing the device. This mode can be used when the user, for example, engages a mute functionality or for when the device is being used to purely collect shoulder angle data.

In the present nonlimiting example, the button mounted to the TPU container 324 on the left side of the flex sensor 301 can turn the device on and off. When it is pressed, the device can be powered on and can begin to function using the power of a portable power supply, such as an attached battery. For example, a battery capacity can be 2500 mAh and can provide around 3.7 volts to the Feather M0 adalogger via a two pin JST connection. This battery can be large enough to power the prototype for an entire day and can be recharged through the Feather. The MCU 303 used in the prototype can have a microUSB connector to upload code and recharge the battery attached to the built in JST connector. It can also feature an assortment of pin connections to connect the various components to and a microSD memory card slot so data can be logged.

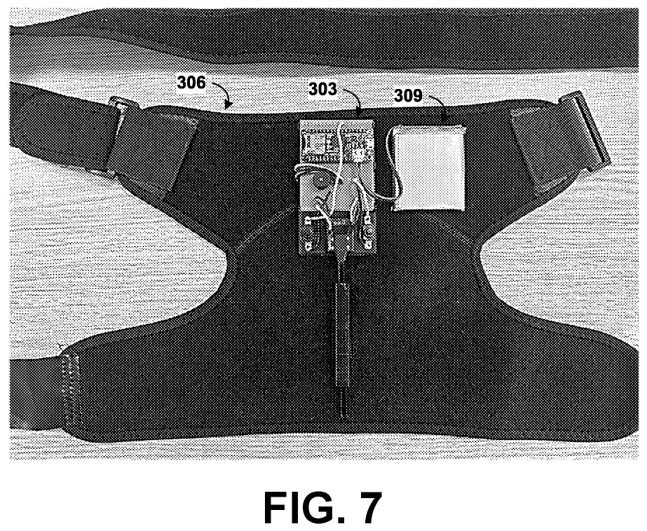

Referring next to FIG. 7, shown is an example of an assembled exemplary prototype according to various examples of the present disclosure. The device module can be mounted on a wearable member in contact with the user where shoulder angle can be accurately measured. In the present nonlimiting example, the device module can be mounted onto a shoulder brace 306, for example, a shoulder bracer 306 from Copper Compression® made from the soft loop side of Velcro® and can be worn on either shoulder of the user. This can allow for the device module to be mounted to the shoulder brace 306 using the rough hook side of Velcro® that can be attached to the bottom of the TPU container 324. A sensor guide 321 can be printed out of TPU and attached by Velcro® to the brace to guide the flex sensor 301 along the shoulder. The sensor guide 321 can define a channel that constrains the sensor 301 along a fixed path during arm movement. This can be to keep the flex sensor 301 on a fixed path but allow it to move with the arm to maintain range of motion and record uniform data. The battery 309 can also be attached by Velcro® directly to the shoulder brace 306. The use of Velcro® to attach components can allow the device to be customized based on the user's anatomy and increases the comfort of the user. The components comprising the exemplary prototype are shown in FIG. 3C.

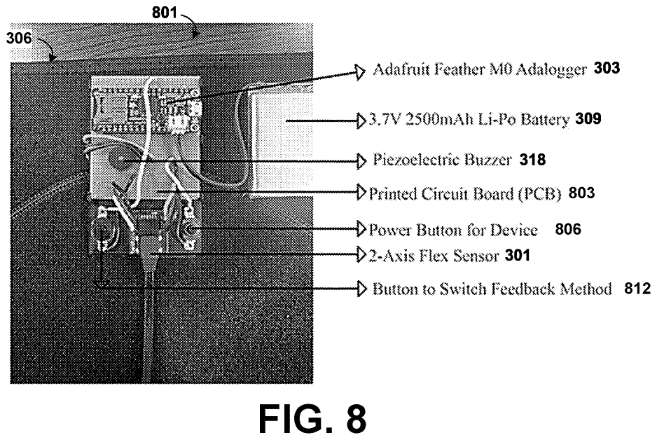

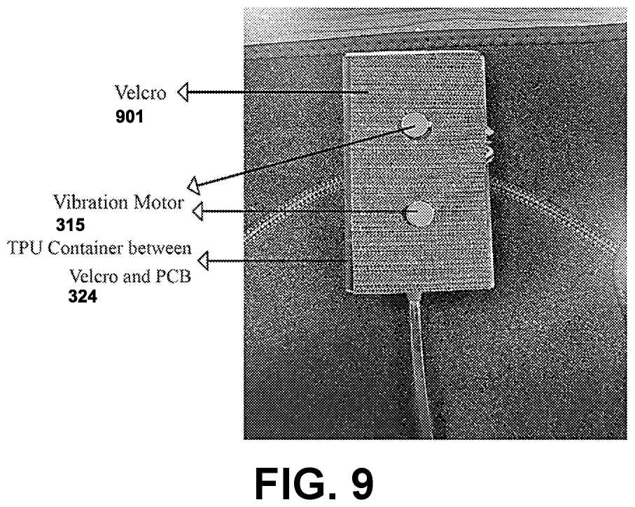

Referring next to FIGS. 8 and 9, shown are a top side and back side view of the exemplary major prototype 801 and its components, respectively, according to various examples of the present disclosure. The front side can show the wiring configurations for each individual component into the PCB. Along with the two buttons (a power button 806 and a button to switch feedback method 812) and the flex sensor 301, the PCB and wiring can be placed within the TPU container 324 so that it could be held in a fixed position. On the opposite side of the TPU container 324, the two vibration motors 315 can be glued to the back side to make it as close to the brace as possible to ensure that vibrations can be felt by a user through the brace. Additionally, a strip of Velcro® 901 can be placed on the back side to allow the mounting device to be placed on the outside of the shoulder brace 306. Not shown in either of the images above is the 3D printed rectangular flex sensor guide 321 that was used to ensure that the sensor 301 traveled along a consistent path during all user arm movements.

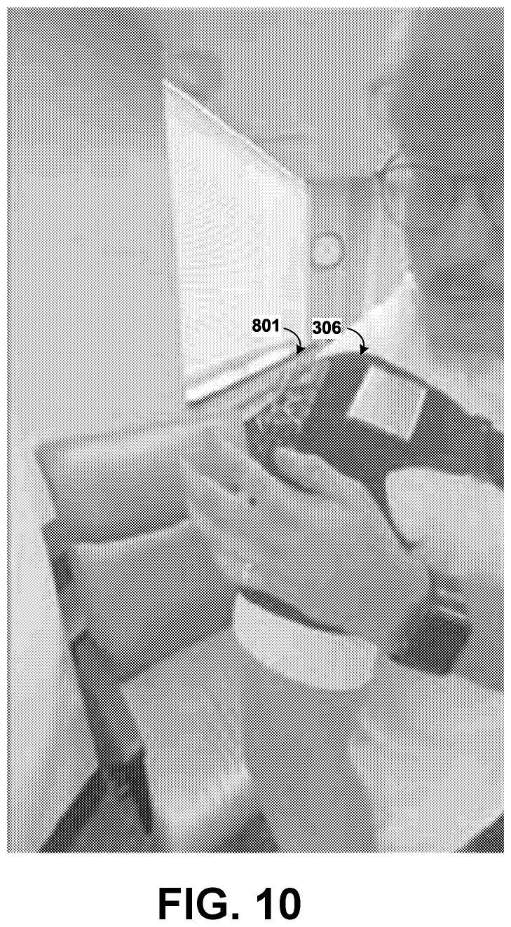

Referring next to FIG. 10, shown is an example of the exemplary prototype 801 worn by a user according to various examples of the present disclosure. The user shown in FIG. 10 has the device placed on their right shoulder. The exemplary device shown in FIG. 10 includes a the power button 806 that, when pressed can turn the device on and the button to switch feedback methods 812 that can be pressed can be used to switch between feedback options. For haptic feedback only, the button to switch feedback methods 812 not need to be pressed, for auditory, the button to switch feedback methods 812 can be pressed once, for both haptic and auditory this button to switch feedback methods 812 can be pressed twice, and to mute all feedback, the button to switch feedback methods 812 can be pressed three times. The various non-mute feedback options can all function to alert the user when they have reached above shoulder height 201. Additionally, a delay can be set to ensure that the device does not beep persistently.

In some examples, the prototype can provide feedback only when the arm is above 90°. Further, in some example, the feedback method can be switched using the button located to the right. The button can be pressed to cycle through feedback methods while performing arm movements above 90°to confirm that the haptic vibration, auditory, both, and no feedback options worked when selected.

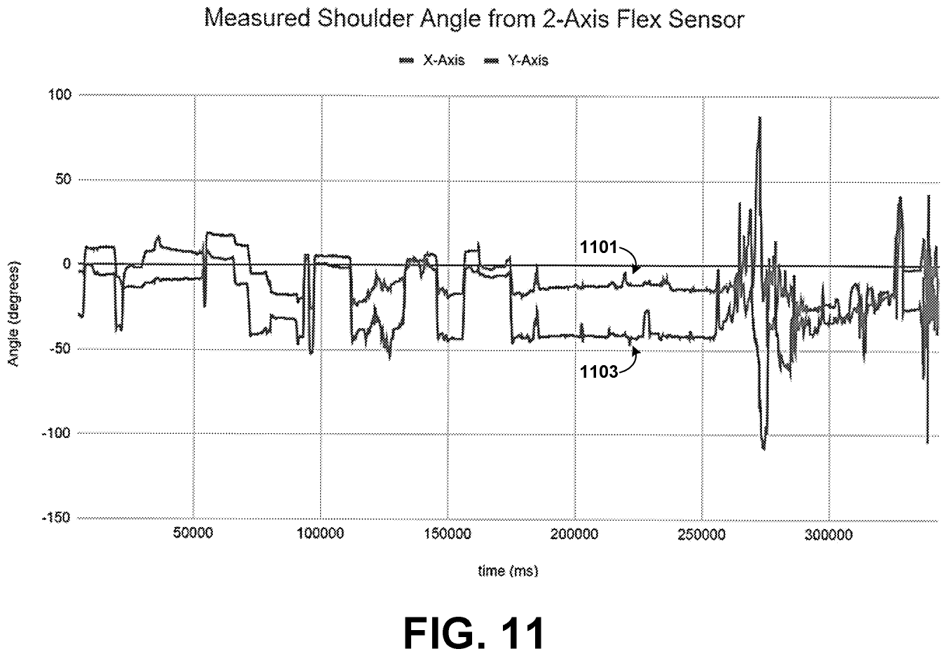

Referring next to FIG. 11, shown is an example graph showing the shoulder angle over a duration of five minutes measured by the flex sensor 301 in two orthogonal (e.g., x-axis 1101 and the y-axis 1103) axes, according to the various examples of the present disclosure. The data collection of the prototype can also tested by having a user wear the device under normal circumstances and log the data to a microSD card. The device's primary focus is to measure the vertical shoulder angle using the y-axis 1103, but both the x-axis 1101 and y-axis 1103 along with the time the measurement can be taken and logged to the microSD card. This can be enabled by, for example, an MCU 303 (e.g., the Feather M0 Adalogger) having a built-in slot for a microSD memory card 312 which can be positioned to be easily accessible for either the user or a professional to retrieve the data. In some examples, the code to log the data can create a new file each time the device is powered on to organize it into different sessions. In one example, an eight gigabyte microSD memory card 312 can be included to store data which can contain plenty of storage due to the small file size of the exported data. The code and microSD memory card 312 can allow for the data to be organized and analyzed by a computer program to view the user's arm motion throughout the day. In FIG. 11, a graph of the shoulder angle in the x-axis 1101 and the y-axis 1103 is shown over a period of about five minutes where the user was performing everyday tasks. With a large battery capacity, the prototype can measure this data over an entire day and record data on small individual session files over weeks using the large microSD memory card 312.

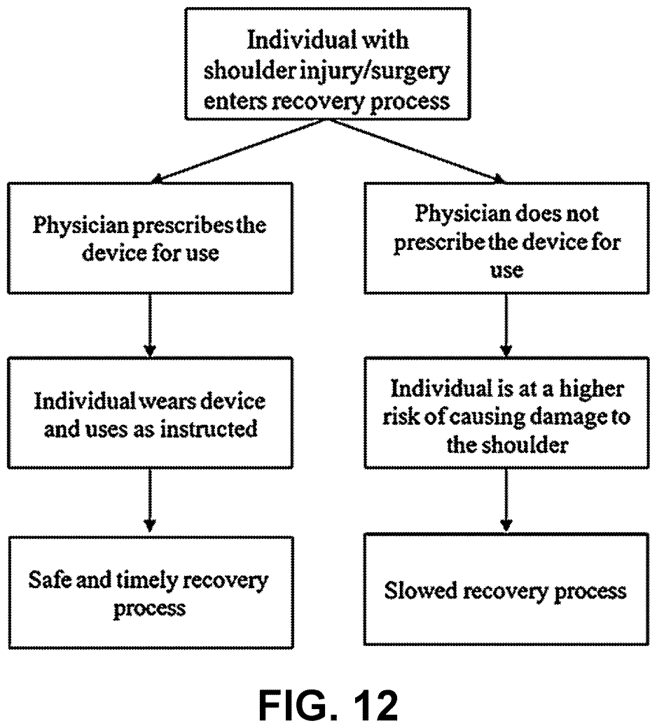

In the present nonlimiting example, the device's cycle of care can be described as follows, shown in FIG. 12. A path to prevent injury can be to administer this type of device to workers at facilities that are required to perform overhead movements throughout their day. Another cycle of care can also be considered. Once an individual experiences a shoulder injury/surgery and enters the recovery process, a physician can choose to either prescribe the device or not. If the physician chooses not to, the individual can have a higher probability of causing damage to the shoulder and slowing down the recovery process. If the physician has the patient wear the device as intended, it can help reduce the risk of causing more damage to the shoulder.

The casing of the device can be altered to have smoothed edges, along with a varied design to result in a more seamless appearance with the shoulder. The attachments located on the edges can also be altered to condense the device more thoroughly to prevent intrusive obstruction of the user's daily movements.

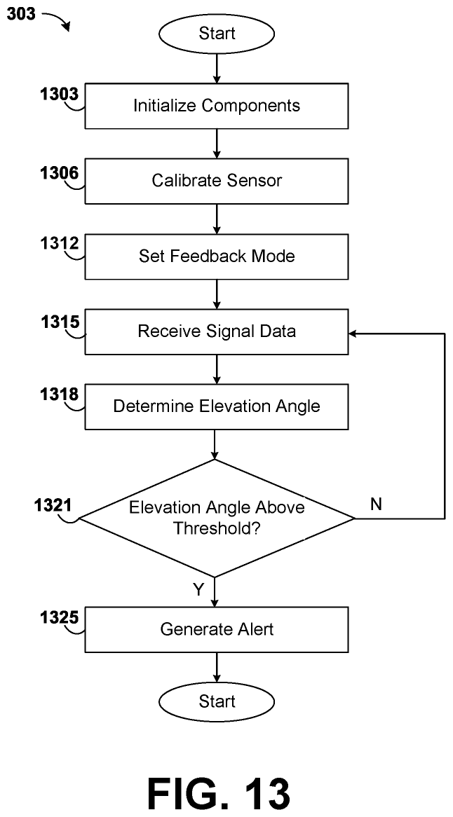

Referring next to FIG. 13, shown is an example flowchart demonstrating the functionality of MCU 303 according to various examples of the present disclosure. In some examples, the MCU 303 can execute computer program software for shoulder elevation measurement. The MCU 303 can be programmed using, for example, the software of Arduino IDE to give the device the main functionality of measuring shoulder angle and alerting the user when their arm is in a position for increased risk.

In the present nonlimiting example, the software for shoulder elevation measurement can be code stored in a memory and executable to perform the various examples of the present disclosure. In some examples, the code can be broken down into the three main sections to initialize variables and code libraries: (1) setup code to only run once on device start up; (2) loop code that can continuously run while the device is powered on; (3) and functions that can be called while the code is running.

In step 1303, the MCU 303 can initialize variables needed for pin locations, feedback and frequency of feedback, and the angle variables with the set angle threshold for feedback. This section can also allow outside libraries to be included into the code, such as the SPI and SD libraries for the microSD memory card 312, and the Arduino and ads_two_axis library to operate the flex sensor 301. The code can be executed to initialize the components and confirm that everything functions and starts up correctly. It can first set the mode of the MCU 303 pins to send or receive data. It can then check to see if a microSD memory card 312 is inserted and can be written to, and can follow by creating a unique file for the current session. After that, it can check battery charge and read the state of the feedback switching button to align the variables related to it in the code. The last step can be to initialize and start the flex sensor 301 with a one second haptic pulse to confirm device startup.

In step 1306, the MCU 303 can calibrate the sensor 301. In some examples, calibrating the sensor can involve establishing an initial reference position when the arm is at rest, such that later measurements can be determined relative to this reference. In some examples, other means of calibrating the sensor 301 can be used.

In step 1312, the MCU 303 can set the feedback mode. A void loop section can provide the main function of the code and this block can be repeated while the device is powered on. It can start by reading the state of the feedback button to change the variable associated with the selected feedback method. It can give confirmation of the switch in the selected feedback method with, for example, a short quarter of a second pulse.

In step 1315, the MCU 303 can receive signal data. In some examples, the MCU 303 can receive the signal data from a two-axis flex sensor 301. In these examples, the x and y axis angle data can then read from the flex sensor 301 and be written to the microSD memory card 312 along with the time the data was collected. In some examples, the MCU 303 in the prototype can not have a clock module, so time can be measured in milliseconds since the device was started.

In step 1318, the MCU 303 can determine a shoulder elevation angle based on the signal data. In some examples, the MCU 303 can determine the elevation angle based on the calibration completed in step 1306, e.g., by comparing the x and y axis angle data from the signal data to the initial reference position when the arm is at rest. In some examples, the MCU 303 can determine a shoulder elevation angle based on the comparison.

In decision step 1321, the MCU 303 can determine whether the elevation angle is above a threshold. In some examples, the threshold can be a pre-defined shoulder elevation angle. After the angle reading, the code can determine if feedback is given based on if the y-axis angle is above 90°and if it has been greater than five seconds since the last angle feedback. If the elevation angle determined in step 1318 is at or above the threshold, the process can continue to step 1325. If the elevation angle is determined in step 1318 to be below the threshold, the process can return to step 1315.

In step 1325, the MCU 303 can generate an alert. In some examples, the feedback can be two short pulses of haptic and/or auditory alerts based on the selected user preference determined in step 1312. For example, delays can be added so the user gets notified but doesn't have constant vibration or audio which could distract them from their current task. Other configuration of notifications and timing can be configurable depending on user preference. In some examples, a part of the loop can check the charge of the battery 309 every fifteen minutes. The fourth section of code can contain various functions which process the flex sensor 301 data, calibrate the flex sensor 301, check the battery charge when called, or can contain other functions.

Those skilled in the art will appreciate that the various examples of the present disclosure may be implemented as any suitable computing environment including a processor programmed to execute software routines adapted to measure and evaluate data as described herein as well as to process and provide feedback to a user. Those skilled in the art will appreciate that the described methodology includes a machine or computer programmed by computer-executable instructions, such as program modules, so as to implement the methods described herein. The computer may be a client workstation, a server, a personal computer, or the like. Generally, the program modules include routines, programs, objects, components, data structures and the like that perform particular tasks or implement particular abstract data types. Moreover, it should be appreciated that the described methodology and/or portions thereof may be practiced with other computer system configurations, including hand-held devices, multi-processor systems, microprocessor-based or programmable consumer electronics, network PCs, minicomputers, mainframe computers and the like. The described methodology may also be practiced in distributed computing environments where tasks are performed by remote processing devices that are linked through a communications network. In a distributed computing environment, program modules may be located in both local and remote memory storage devices.

Further, stored in a memory can be both data and several components that are executable by the processor. In particular, stored in the memory and executable by the processor 2013 are code and potentially other applications. Also stored in the memory may be a data store and other data. In addition, an operating system may be stored in the memory and executable by the processor. It is understood that there may be other applications that are stored in the memory and are executable by the processor as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages. A number of software components are stored in the memory and are executable by the processor. In this respect, the term “executable” means a program file that is in a form that can ultimately be run by the processor. Examples of executable programs may be, for example, a compiled program that can be translated into machine code in a format that can be loaded into a random access portion of the memory and run by the processor, source code that may be expressed in proper format such as object code that is capable of being loaded into a random access portion of the memory and executed by the processor, or source code that may be interpreted by another executable program to generate instructions in a random access portion of the memory to be executed by the processor, etc.

Those skilled in the art will further appreciate that the computer may further include a device (not shown) for reading computer readable storage media such as a hard disk, a removable magnetic disk, and/or a removable optical disk such as a CD-ROM or other optical media. Such devices (e.g., hard disk drive) and their associated computer-readable storage media provide non-volatile storage of computer readable instructions, data structures, program modules and other data for the computer. It will be further appreciated by those skilled in the art that other types of computer readable media that can store data that is accessible by a computer may also be used to store software code for implementing the described methodology in the exemplary operating environment. Such other types of media include a magnetic cassette, a flash memory card, a digital video disk, a Bernoulli cartridge, a random access memory (RAM), a read-only memory (ROM), and the like. The described methodology includes such computer readable storage media having instruction stored thereon that, when executed by a processor, cause the processor to implement the techniques of the methodology as described.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

1. A system, comprising:
a wearable support configured to be positioned on a shoulder of a user;
at least one sensor coupled to the wearable support;
a processing unit operatively coupled to the at least one sensor; and
at least one feedback device operatively coupled to the processing unit;
wherein the processing unit is configured to:
determine, based on signal data received from the at least one sensor, a shoulder elevation angle;
compare the shoulder elevation angle to a predefined elevation threshold; and
in response to determining that the shoulder elevation angle exceeds the predefined elevation threshold, cause the at least one feedback device to generate an alert.

2. The system of claim 1, wherein the at least one feedback device is haptic feedback, auditory feedback, or a combination thereof.

3. The system of claim 1, further comprising at least one portable power source.

4. The system of claim 1, wherein the at least one sensor is a two-axis flex sensor.

5. The system of claim 1, wherein the alert is a first alert, and wherein the processing unit a generates a second alert when the shoulder elevation angle continues to exceed the predefined elevation threshold for a predefined period of time.

6. The system of claim 1, wherein the processing unit is a microcontroller unit (MCU), and wherein the MCU records the shoulder elevation angle.

7. The system of claim 6, further comprising a memory, wherein machine-readable instructions stored in the memory cause the MCU to store the shoulder elevation angle and a timestamp in the memory.

8. The system of claim 1, wherein the predefined elevation threshold is about 90 degrees.

9. A method, comprising:
initializing, by a processing unit, at least one sensor coupled to a wearable support;
receiving, by the processing unit, signal data corresponding to angular displacement of an arm of a user relative to a shoulder of the user; and
in response to determining that a shoulder elevation angle exceeds a predefined threshold based, causing, by the processing unit, at least one feedback device to generate an alert.

10. The method of claim 9, further comprising:
storing, by the processing unit, the signal data in a memory.

11. The method of claim 9, wherein determining that the shoulder elevation angle exceeds the predefined threshold further comprises comparing the signal data corresponding to the angular displacement of the arm of the user relative to the shoulder of the user to a baseline measurement.

12. The method of claim 9, wherein the alert is a first alert, and wherein the processing unit a generates a second alert when the shoulder elevation angle continues to exceed the predefined threshold for a predefined period of time.

13. The method of claim 9, wherein the alert is a haptic alert, auditory alert, or a combination thereof.

14. A method, comprising:
receiving, by a processing unit, signal data from at least one sensor coupled to a wearable support;
determining, by the processing unit, a shoulder elevation angle based at least in part on the signal data;
comparing, by the processing unit, the shoulder elevation angle to a predefined elevation threshold; and,
in response to determining that the shoulder elevation angle exceeds the predefined elevation threshold, generate an alert.

15. The method of claim 14, wherein generating the alert comprises sending a signal to at least one feedback device, and wherein the at least one feedback device is a haptic feedback device, an auditory feedback device, or a combination thereof.

16. The method of claim 14, wherein the at least one sensor is a flex sensor.

17. The method of claim 14, wherein the alert is a first alert, and wherein the processing unit a generates a second alert when the shoulder elevation angle continues to exceed the predefined elevation threshold for a predefined period of time.

18. The method of claim 14, wherein the processing unit is a microcontroller unit (MCU), and wherein the MCU records the shoulder elevation angle.

19. The method of claim 14, and wherein the processing unit is configured to store the shoulder elevation angle and a timestamp in a memory.

20. The method of claim 14, wherein the predefined elevation threshold is about 90 degrees.
